(19) United States
(12) Patent Application Publication
(10) Pub. No.: US 2014/0214004 A1
(43) Pub. Date: Jul. 31, 2014

(54) Intravascular Devices Having Artificial Muscles and Associated Systems and Methods

(71) Applicant: Volcano Corporation, San Diego, CA (US)

(72) Inventor: Bret C. Millett, Folsom, CA (US)

(73) Assignee: Volcano Corporation, San Diego, CA (US)

(21) Appl. No.: 14/139,500

(22) Filed: Dec. 23, 2013

Related U.S. Application Data
(60) Provisional application No. 61/747,958, filed on Dec. 31, 2012.

(51) Int. Cl. A61M 25/01; A61B 17/3207; A61B 17/00

(52) U.S. Cl. CPC A61M 25/0158; A61B 17/00234; A61B 17/3207
USPC 604/510; 604/95.05

(57) ABSTRACT

Intravascular devices, systems, and methods are disclosed. In some embodiments, the intravascular device includes at least one artificial muscle segment. In some instances, the artificial muscle segment or a plurality thereof is configured to move a distal portion of the intravascular device, change the shape of the tip of the intravascular device, rotate a portion of the intravascular device, and any combination thereof. A method for using an associated intravascular device is also provided. The method includes inserting an intravascular device into a vessel of a patient. The intravascular device has an actuator segment. The method further includes moving a portion of the intravascular device by selectively rotating the actuator segment and returning the portion of the intravascular device to a pre-activation position by selectively deactivating the actuator segment.

### CROSS-REFERENCE TO RELATED APPLICATIONS

The present application claims priority to and the benefit of U.S. Provisional Patent Application No. 61/747,958, filed Dec. 31, 2012, which is hereby incorporated by reference in its entirety.

### TECHNICAL FIELD

The present disclosure relates to intravascular devices, systems, and methods utilized in the performance of medical procedures, including both diagnostic and therapeutic procedures.

### BACKGROUND

Given the many uses of such intravascular devices, there are many different kinds that are suited to many different situations. For example, while guiding an intravascular device into a desired region of interest, a straight tip is ideal for following a main line, while a J-shaped tip may be preferable for navigating into a side-branch. As another example, a flexible tip is preferable for advancing an intravascular device through tortuous vasculature, while a rigid tip is ideal when advancing through a chronic total occlusion (CTO). Additionally, a more rounded tip is preferable in some circumstances while a more pointed tip is preferable in others.

However, in each of these circumstances, the selection must be made before the intravascular device is inserted into a patient. Regardless of the selection, it will be less than ideally suited for at least a portion of most procedures.

Furthermore, current intravascular devices permit rotation of an intravascular device or a drive shaft of the intravascular device by rotating a proximal end of the device or drive shaft. However, depending on the inserted distance and the tortuous path of the device, the rotation resulting on the distal end varies from that imparted at the proximal end. For example, non-uniform rotational distortion in some instances.

Accordingly, there remains a need for improved intravascular devices, systems, and methods that include one or more electronic, optical, or electro-optical components.

### SUMMARY

Embodiments of the present disclosure are directed to intravascular devices, systems, and methods.

In one embodiment, an intravascular device is provided. The intravascular device comprising a flexible elongate member with a proximal portion and a distal portion and at least one torsional actuator segment. The torsional actuator segment is coupled to the flexible elongate member and has a first and a second end. The first end of the torsional actuator segment rotates along an axis of the flexible elongate member relative to a second end of the torsional actuator segment when the torsional actuator segment is supplied with an activation energy.

In another embodiment, another intravascular device is provided. This other intravascular device comprises a flexible elongate member having a distal end and a proximal end and a morphable tip at the distal end. The morphable tip has a first shape when the tip is in a dormant state and a second shape when in an activated state.

In another embodiment, a method for using such an intravascular device is also provided. Such a method includes a plurality of steps. One step of the method is inserting the intravascular device into a vessel of a patient, the intravascular device having a torsional actuator segment, and another is rotating a portion of the intravascular device by activating the torsional actuator segment.

In an embodiment, another method for using an intravascular device. The method includes inserting the intravascular device into a vessel of a patient, positioning the intravascular device at a region of interest, and activating an artificial muscle segment to apply an associated treatment at the region of interest.

In one embodiment, a method for using an intravascular device includes inserting an intravascular device into a vessel of a patient. The intravascular device has an actuator segment. The method further includes moving a portion of the intravascular device by selectively activating the actuator segment and returning the portion of the intravascular device to a pre-activation position by selectively deactivating the actuator segment.

Additional aspects, features, and advantages of the present disclosure will become apparent from the following detailed description.

### BRIEF DESCRIPTION OF THE DRAWINGS

Illustrative embodiments of the present disclosure will be described with reference to the accompanying drawings, of which:

FIG. 1A provides a pair of diagrammatic, schematic side views of an artificial muscle element according to an embodiment of the present disclosure.

FIG. 1B provides a pair of diagrammatic, schematic side views of an additional artificial muscle element according to an embodiment of the present disclosure.

FIG. 2A provides a pair of diagrammatic side views of an intravascular device similar having activated and dormant states according to an embodiment of the present disclosure.

FIG. 2B is a diagrammatic end view of the intravascular device of FIG. 2A as seen along the arrow 2B.

FIG. 2C is a diagrammatic end view of the intravascular device of FIG. 2A as seen along the arrow 2C.

FIG. 3A is a diagrammatic side view of an intravascular device incorporating at least one artificial muscle element according to an embodiment of the present disclosure.

FIG. 3B is a diagrammatic side view of an intravascular device incorporating at least one artificial muscle element similar to that of FIG. 3A, but illustrative of another embodiment of the present disclosure.

FIG. 3C is a diagrammatic side view of an intravascular device incorporating at least two artificial muscle elements according to an embodiment of the present disclosure.

FIG. 4A is a diagrammatic side view of an intravascular device including a plurality of morphable tip according to an embodiment of the present disclosure. Please add the following new paragraphs following paragraph

FIG. 4B is a diagrammatic side view of an intravascular device including a morphable tip according to another embodiment of the present disclosure.

FIG. 4C is a diagrammatic side view of an intravascular device including a morphable tip according to another embodiment of the present disclosure.

FIG. 5A is a diagrammatic end view of a morphable segment of an intravascular device in a first state according to some embodiments of the present disclosure.

FIG. 5B is a diagrammatic end view of a morphable segment of the intravascular device of FIG. 5A in a difference state according to an embodiment of the present disclosure.

FIG. 5C is a diagrammatic end view of a morphable segment of the intravascular device of FIG. 5A in a different state according to another embodiment of the present disclosure.

FIG. 6 provides a pair of diagrammatic end views of an intravascular device in two different states positioned within a vessel in two different states utilized to center the intravascular device within the vessel according to an embodiment of the present disclosure.

FIG. 7A provides a pair of diagrammatic side views of an intravascular device having a dynamic portion at its distal end.

FIG. 7B provides a pair of diagrammatic side views of an intravascular device having a dynamic portion.

FIG. 7C provides a pair of diagrammatic side views of an intravascular device having a dynamic portion at its distal end according to another embodiment.

FIG. 8 provides a pair of diagrammatic perspective views of an intravascular device having a rotating tip in an activated and a dormant state according to an embodiment of the present disclosure.

FIG. 9 provides a pair of diagrammatic perspective views of an intravascular device having a rotating segment located near the tip in an activated and a dormant state according to an embodiment of the present disclosure.

FIG. 10 is flow chart of a method of using an intravascular device having an actuator segment according to an embodiment of the present disclosure.

FIG. 11 is flow chart of another method of using an intravascular device having an actuator segment according to an embodiment of the present disclosure.

For clarity of discussion, elements having the same designation in the drawings may have the same or similar functions. The drawings may be better understood by referring to the following Detailed Description.

### DETAILED DESCRIPTION

For the purposes of promoting an understanding of the principles of the present disclosure, reference will now be made to the embodiments illustrated in the drawings, and specific language will be used to describe the same. It is nevertheless understood that no limitation to the scope of the disclosure is intended. Any alterations and further modifications to the described devices, systems, and methods, and any further application of the principles of the present disclosure are fully contemplated and included within the present disclosure as would normally occur to one skilled in the art to which the disclosure relates. In particular, it is fully contemplated that the features, components, and/or steps described with respect to one embodiment may be combined with the features, components, and/or steps described with respect to other embodiments of the present disclosure. For the sake of brevity, however, the numerous iterations of these combinations will not be described separately.

As used herein, “flexible elongate member” or “elongate flexible member” includes at least any thin, long, flexible structure that can be inserted into the vasculature of a patient. While the illustrated embodiments of the “flexible elongate members” of the present disclosure have a cylindrical profile with a circular cross-sectional profile that defines an outer diameter of the flexible elongate member, in other instances all or a portion of the flexible elongate members may have other geometric cross-sectional profiles (e.g., oval, rectangular, square, elliptical, etc.) or non-geometric cross-sectional profiles. Flexible elongate members include, for example, guidewires and catheters. In that regard, catheters may or may not include a lumen extending along its length for receiving and/or guiding other instruments. If the catheter includes a lumen, the lumen may be centered or offset with respect to the cross-sectional profile of the device.

“Connected”, “coupled”, and variations thereof as used herein includes direct connections, such as being glued or otherwise fastened directly to, on, within, etc. another element, as well as indirect connections where one or more elements are disposed between the connected elements.

“Intravascular device”, as used herein includes hypotubes, catheters, guidewires, and other devices such as may be used by a doctor to provide various treatments, and to obtain data from vessels throughout the body.

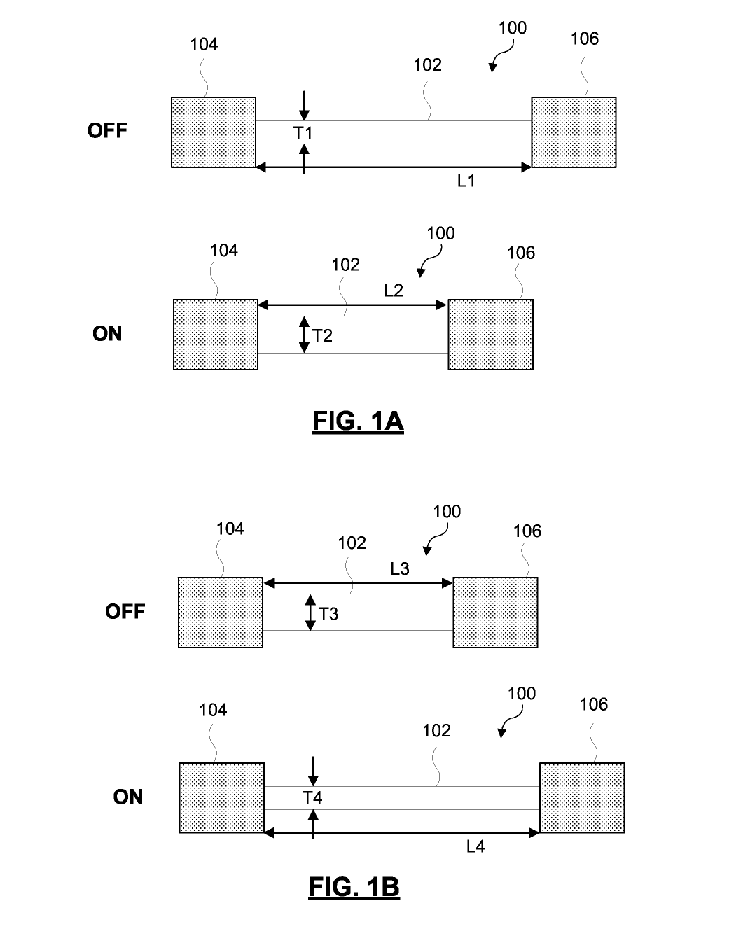

Referring now to FIG. 1, therein is a schematic depiction of an artificial muscle system 100 that includes an artificial muscle 102 in an on (i.e., activated) state and in an off (i.e., dormant) state as labeled. FIG. 1A illustrates how some artificial muscles operate in accordance with the present disclosure. FIG. 1A includes an attachment site 104 and an attachment site 106 being coupled to the proximal and distal ends of the artificial muscle 102, respectively. As depicted, the artificial muscle 102 has a length L1 and a thickness T1 while in the dormant state.

When an activation energy is applied to the artificial muscle 102, attachment site 106 moves closer to attachment site 104 as a result of the change in shape of the artificial muscle 102. Thus, as depicted in FIG. 1A, when the activation energy is applied to the artificial muscle 102, the activated length L2 decreases. At the same time, the activated thickness T2 increases relative to the dormant thickness T1.

FIG. 1B depicts many features similar to those of FIG. 1A, but illustrating how other artificial muscles operate in accordance with embodiments of the present disclosure. Thus, FIG. 1B depicts an artificial muscle system 100 in activated and dormant states. The artificial muscle system 100 includes an artificial muscle 102 coupled to attachment sites 104 and 106. The attachment sites are able to move relative to each other in response to the changes in the shape of the artificial muscle 102. In the dormant state, the artificial muscle 102 of FIG. 1B has a thickness T3 and a length L3.

When an activation energy is applied to the artificial muscle 102, the distance between the attachment site 104 and the attachment site 106 increases, such that the activated length L4 is greater than the dormant length L3. As a result, the distance between the attachment sites 104 and 106 also increases when activated. The thickness of the artificial muscle decreases. Thus, the activated thickness T4 is less than the dormant thickness T3.

In general, the artificial muscle 102 changes its dimensions when an activation energy is applied, and the change in dimensions can be used to perform work of various kinds in the context of the intravascular device of the present disclosure. This can be used for drilling, sawing, pounding, and otherwise removing material from the surface of a vessel. Various materials may be used to provide the artificial muscle. In the depicted embodiments of FIGS. 1A and 1B, the artificial muscle 102 is an electroactive polymer. Suitable electroactive polymers include polyetherimide, polyetheretherketone, polyethylene, polyamides, liquid crystal polymers, as well as many other materials, and combinations thereof. As illustrated in FIGS. 1A and 1B, in some embodiments, the application of an activation energy causes a length of the artificial muscle 102 to contract, while in others it causes the length of the artificial muscle 102 to contract. In some embodiments, the artificial muscle 102 may be made from carbon nanotube yarn.

Additionally, the activation energy applied to the artificial muscle 102 in FIG. 1A or 1B can be supplied in a variety of ways. In the depicted embodiments, a voltage is applied on either end of the artificial muscle. Thus in some instances, the attachment sites 104 and 106 are conductive and coupled to wires that supply voltage and extend along the length of the intravascular device into which the artificial muscle 102 is incorporated. These electrically responsive electroactive polymers can be ferroelectric polymers, dielectric polymers or others. In some embodiments, the activation energy is provided by interactions with chemical ions. In such embodiments, the electroactive polymer used is an ionic electroactive polymer. Additional sources of activation energy in some embodiments are light and/or heat. In many embodiments, the degree of change is controllable, such as by controlling the voltage, light, and/or heat applied. In such embodiments the response of the electroactive polymer can be carefully controlled by controlling the amount of activation energy applied and the way in which is applied. In some instances this activation energy is determined by a controller in communication with the intravascular device and/or other medical components. Information may be received from other medical components and taken into consideration when determining the activation energy to be applied.

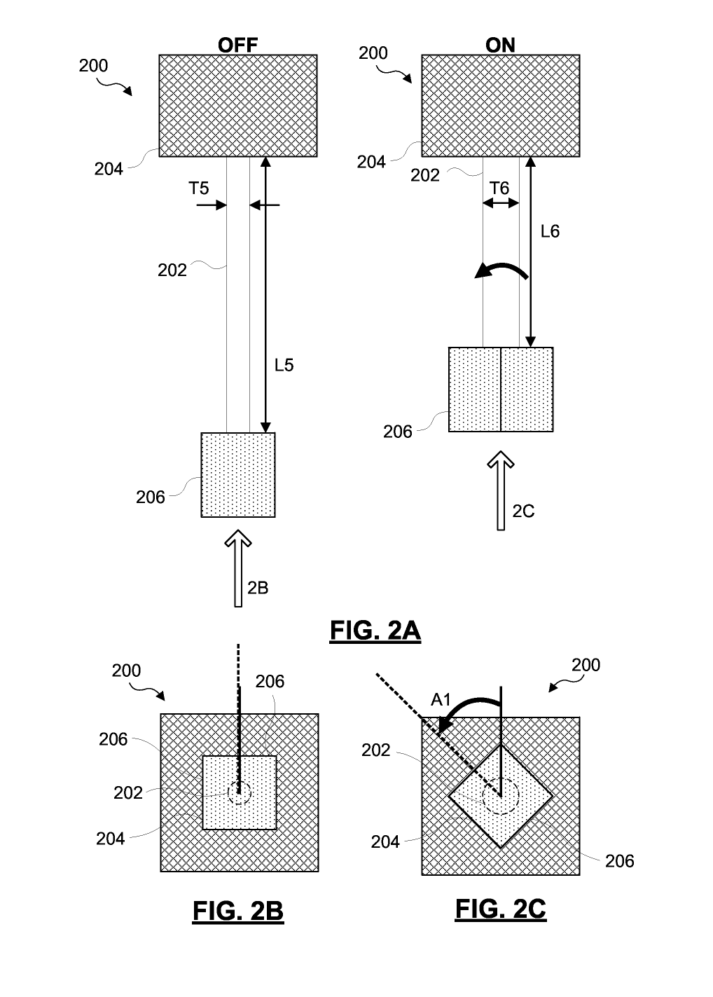

Referring to FIGS. 2A, 2B, and 2C, a torsional actuator system 200 is depicted. While the artificial muscles 102 of FIGS. 1A and 1B may be thought of as linear actuators. FIG. 2A depicts an artificial muscle 202 that rotates in response to an activation energy. As depicted, the artificial muscle 202 is coupled to an attachment site 204 on one end and an attachment site 206 on the other end. In FIGS. 1A and 1B, the attachment sites 104 and 106 are illustrated as being substantially the same size. In FIGS. 2A, 2B, and 2C, the attachment sites 204 and 206 are illustrated as differently sized from each other in order to more clearly convey some embodiments. In general, any combination of sizes of attachment sites may be used.

As depicted on the left in FIG. 2A, the torsional actuator system 200 is in an OFF or dormant state. In the dormant state, the artificial muscle has a length L5 and a thickness T5. An end view of the torsional actuator system 200 as seen along arrow 2B, is illustrated in FIG. 2B. When an activation energy is applied, as depicted on the right in FIG. 2A, the artificial muscle 202 rotates as illustrated by the rotation of attachment site 206. In some embodiments, due to the rotation and/or in conjunction with the rotation, the distance between the attachment sites 204 and 206 decreases to length L6. At the same time, the thickness of the artificial muscle 202 increases to thickness T6.

FIG. 2C illustrates a view of the torsional actuator system in an activated state as seen looking in the direction of arrow 2C of FIG. 2A. The rotation of the attachment site 206 is illustrated in FIG. 2C. As depicted, the artificial muscle 202 that couples the attachment sites 204 and 206 has imparted a relative rotation of attachment site 206 to attachment site 204 as depicted by angle A1. Treating the orientation of attachment site 204 as fixed and attachment site 206 as unfixed for illustrative purposes, attachment site 206 rotates along with the artificial muscle 202 through angle A1, which is depicted as about 45 degrees. However, some embodiments provide more or less rotation. Generally speaking, the angle A1 is between about 10 degrees and 1080 degrees. To this end, the characteristics of the artificial muscle 202 (length, activated or unactived state shapes, applied voltages, materials, etc.) may be selected based on the desired rotation.

Some embodiments of artificial muscle 202 use carbon nanotube yarns to provide the rotation and the corresponding decrease in length of artificial muscle 202. More detail regarding the fabrication and application of carbon nanotube yarns and other nanofiber yarns can be found in US Pat. Pub. No. 2008/0170982 A1, filed on Nov. 9, 2005, which is hereby incorporated by reference in its entirety. Carbon nanotube yarns, electroactive polymers, and other artificial muscles may be used in intravascular device in various applications explained in more detail below.

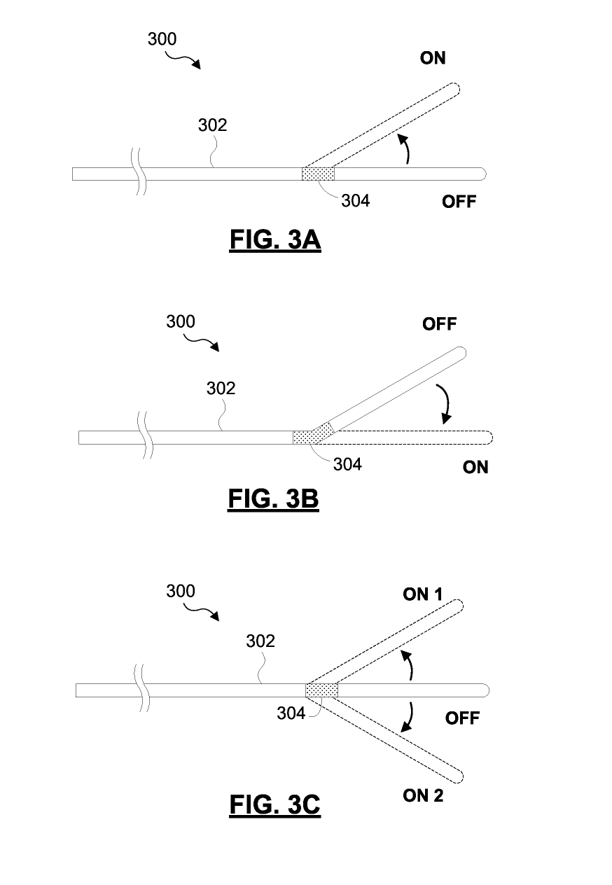

Referring to FIGS. 3A, 3B, and 3C, several embodiments of actuatable intravascular devices are depicted. In FIG. 3A, an intravascular device 300 is depicted that has an activated state and a dormant state. Intravascular device 300 includes a flexible elongate member 302 with proximal and distal portions. As illustrated in FIG. 3A, the flexible elongate member 302 is a generally straight member, with an actuator segment 304. The actuator segment 304 is made from an electroactive polymer that has two wires coupled to it, both wires running the length of the flexible elongate member 302 beyond the proximal portion thereof. In some embodiments, the actuator segment 304 is formed from plurality of artificial muscle strands. The solid lines of the intravascular device 300 depict the device in a dormant state. When an activating voltage is supplied to the actuator segment 304, a bend forms in the flexible elongate member 302 in the area near the actuator segment 304. The bend that results from the activated state is depicted in the dashed lines of FIG. 3A. Selective activation of the actuator segment can be utilized to steer the distal end of the intravascular device along a desired path.

Referring to FIG. 3B, an alternative embodiment of intravascular device 300 is depicted. In the embodiment of intravascular device 300 in FIG. 3B, a flexible elongate member 302 has an existing bend or curve situated at or near an actuator segment 304. Unlike the device of FIG. 3A, this flexible elongate member 302 is bent or curved in its dormant state, as depicted in solid lines. When an activation energy is applied to the actuator segment 304, the bend in the flexible elongate member 302 becomes generally straightened, as depicted in the dashed lines. Thus, the devices of FIGS. 3A and 3B are similar but have opposite configurations given the same state of the actuator segment 304. Selective activation of the actuator segment can be utilized to steer the distal end of the intravascular device along a desired path.

Referring to FIG. 3C, yet another alternative embodiment of intravascular device 300 is illustrated. Similar to those discussed above with respect to FIGS. 3A and 3B, intravascular device 300 of FIG. 3C has a flexible elongate member 302 with an actuator segment 304. While the actuator segments in FIGS. 3A and 3B each had a bending motion in one direction, the actuator segment 304 of FIG. 3C facilitates bending motions in two opposing directions. In general, any type of actuator having two opposed position may be used such that in one activated condition the distal end of the intravascular device 300 moves up, while in another activated condition the distal end moves down.

In the depicted embodiment, the actuator segment includes at least a top artificial muscle and a bottom artificial muscle. When the top artificial muscle or set is activated (depicted as ON 1), the distal portion of the flexible elongate member 302 is bent upwards as shown in the top dashed line. Alternatively, when a bottom artificial muscle or set is activated (depicted as ON 2), the distal portion of the flexible elongate member 302 is bent downwards as shown in the bottom dashed line.

In embodiments in which the artificial muscles expand rather than contract, the top artificial muscle may be used to bend the distal portion downward, and the bottom artificial muscle may be used to bend the distal portion upward. Additionally, the rigidity of the intravascular device 300 may be increased by supplying both the top and bottom artificial muscles with corresponding activation energies. For example, the same voltage may be applied to both artificial muscles. The added stresses counteract each other so that the distal end of flexible elongate member 302 does not move due to the activation energies, but instead is maintained in a stiffened linear orientation. In this way the rigidity of intravascular device 300 can be tailored for different applications while in use within a patient.

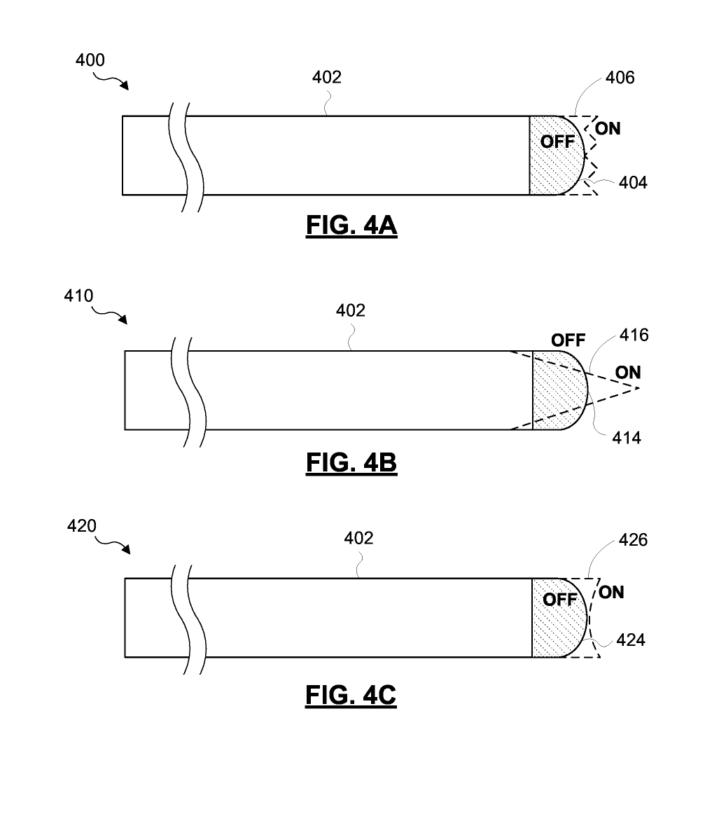

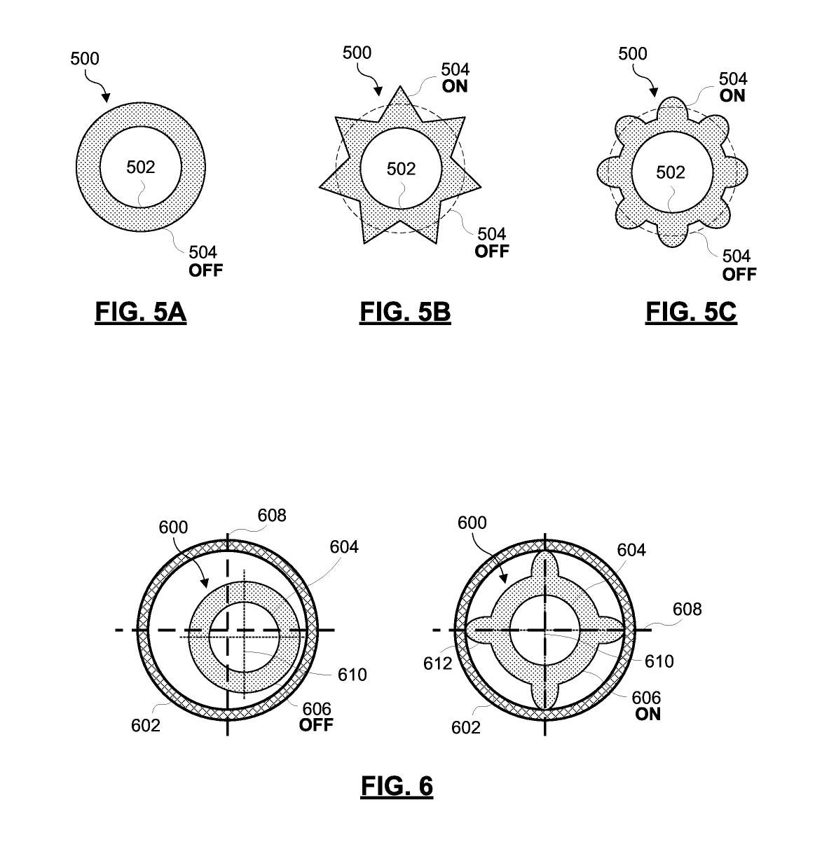

Referring to FIGS. 4A, 4B, and 4C, artificial muscles being used to morph the tip of intravascular devices into various shapes are illustrated. Each of FIGS. 4A, 4B, and 4C includes an intravascular device 400, 410, and 420, respectively, that has a flexible elongate member with a distal portion 402. The distal portion 402 terminates in a distal end. As depicted, the distal end includes a morphable tip 404 made from artificial muscle. As illustrated, the morphable tip 404 has a generally rounded surface that protrudes out somewhat in the center when in a dormant state. However, when an activation energy is applied to the morphable tip 404 it can morph into many different shapes and surfaces depending on how it is configured. For example, morphable tip of intravascular device 400 has a saw-shaped or bumped profile 406 when activation energy is applied as shown in FIG. 4A. This bumped profile 406 has a plurality of rises or bumps that protrude out from the distal end. The change in surface makes the distal end of intravascular device 400 more abrasive and so may be used to remove material in the activated state, while better suited for less abrasive navigation in the dormant state.

FIG. 4B illustrates an intravascular device 410 with a flexible elongate member 402 that has a morphable tip 414 that when activated forms a shape different from that of morphable tip 404 of FIG. 4A. In a dormant state, the morphable tip 414 has a rounded profile, but when an activation energy is applied the morphable tip 414 morphs so that it has the profile 416. Profile 416 is depicted as pointed or chisel-shaped. In some embodiments, the activated profile 416 does not form a point, but has a significantly decreased radius in the curve of the morphable tip 414. This sharpening, pointing, or decreasing in the radius may better suits the intravascular device 410 for use in moving through a barrier such as a chronic total occlusion (CTO). When a CTO is encountered, the morphable tip 414 is activated, sharpening to the profile 416 to better move through the CTO. After the CTO is passed (as well as before it is encountered), the morphable tip 414 is deactivated, because its dormant state is more rounded and may be better able to move through vasculature without unintentionally damaging the walls thereof.

FIG. 4C illustrates an intravascular device 420 which has a morphable tip 424 situated at a distal end of a flexible elongate member 402. The morphable tip 424, when activated, has a shape that is different from the activated shapes of morphable tips 414 and 404 of FIGS. 4B and 4A, respectively. When an adequate activation energy is applied, the artificial muscle or muscles in the tip 424 respond by forming the profile 426. The profile 426 is a concave profile rather than the convex profile to the morphable tip 426 is its dormant state. Similar to the activated profiles 406 and 416 discussed above. The concave profile 426 may allow the intravascular device 420 to dynamically alter its structure so that it is better suited to different applications and/or conditions encountered during the course of a single procedure.

The morphable tips 404, 414, and 424 are made of electroactive polymer in the illustrated embodiments. In such embodiments, at least two wires running from the proximal end of the respective flexible elongate members to the morphable tips to supply them with an activation voltage. In some embodiments, the flexible elongate members depicted in FIG. 4 further include actuator segments like those depicted in FIGS. 1A, 1B, 2A, 2B, 2C, 3A, 3B, and 3C. The morphable tips 404, 414, and 424 allow the tip of an intravascular device to be dynamically reconfigured for multiple purposes. The morphable tips 404, 414, and 424 can be used to optimize the performance of an intravascular device when used for procedures like drilling, sawing, jack-hammering, and otherwise removing material, and for positioning the device. More detail in this regard is included below.

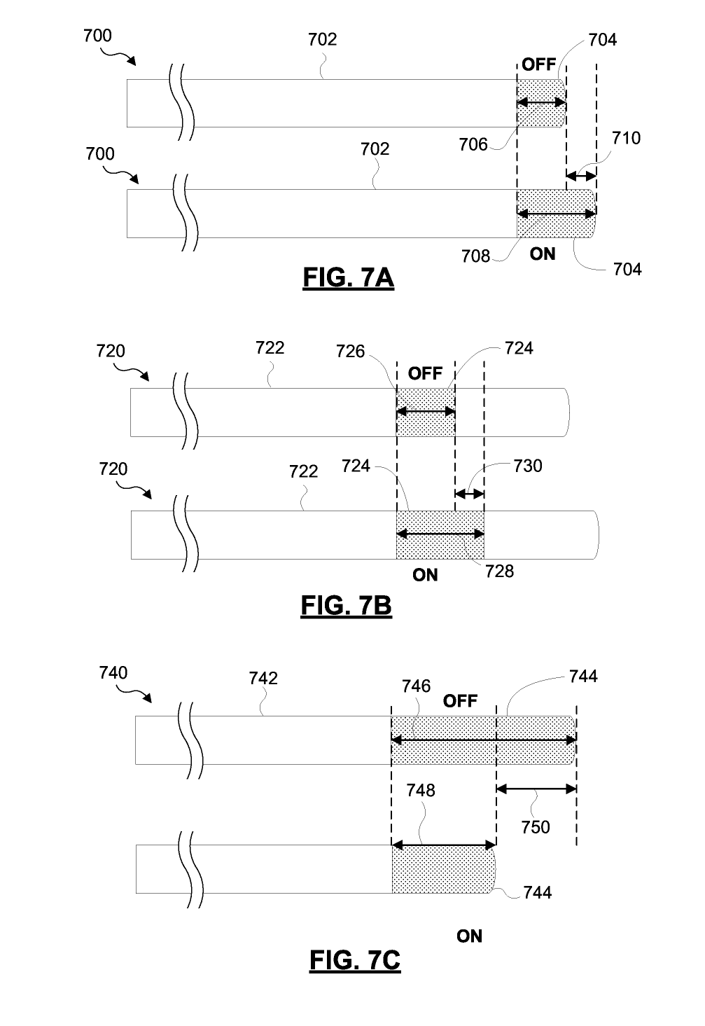

Referring to FIG. 5A, a section 500 of an intravascular device is depicted in cross-section. The depicted cross-section is an axial view of a distal portion of an intravascular device. Some suitable intravascular devices include those depicted in FIGS. 3A, 3B, 3C, 4A, 4B, and 4C. As depicted in FIG. 5A, the section 500 is not located at the distal tip of the intravascular device, however in some embodiments, section 500 is located at the distal tip of an intravascular device. As depicted in FIG. 5A, section 500 includes a static portion 502 that does not include an artificial muscle (i.e., does not change in shape and/or dimensions when exposed to an activation energy). While depicted as solid, in some embodiments this static portion 502 has one or more lumens running through it, such as would allow wires, sensors, guidewires, etc., to pass through. Surrounding the static portion 502 is a dynamic portion 504, which includes one or more artificial muscles as depicted in FIGS. 1A, 1B, 2A, 2B, 2C, 3A, 3B, 3C, 4A, 4B, and 4C and described in corresponding portions of this disclosure. As depicted, the dynamic portion 504 is in a dormant state such that no activation energy is applied to it.

FIG. 5B is a cross-sectional view of the dynamic portion 504 in an activated state depicted relative to the dormant state shown in FIG. 5A (depicted in dashed lines). The activation energy provided to the artificial muscles in the activated dynamic portion 504 cause a change in the external surface of the section 500 of the intravascular device, such that a number of projections are formed. In the illustrated embodiment of FIG. 5B, the projections have angular profiles with pointed tips. This change in the external surface may be used to provide an abrasive surface to facilitate material removal from a site or to temporarily secure the intravascular device in place in a vessel.

FIG. 5C depicts an additional embodiment of a section 500 of an intravascular device depicted in cross-section with the dynamic portion 504 in an activated state. For comparison, the profile of the dynamic portion 504 in a dormant state as depicted in FIG. 5A in shown in dashed lines. FIG. 5C depicts a static portion 502 surrounded by the activated dynamic portion 504. The activated dynamic portion 504 of FIG. 5C differs from the corresponding portion in FIG. 5B in both the shape and geometry of the profile, with the projections the activated dynamic portion 504 having more rounded profiles than the embodiment of FIG. 5B. Many such variations are possible and may be suited for alternative and/or complementary uses. Accordingly, an intravascular device may include a section as depicted in FIG. 5B in addition to a section as depicted in FIG. 5C. Sections include the dynamic portion 504 may be incorporated in intravascular device that also share features depicted in FIGS. 1A, 1B, 2A, 2B, 2C, 3A, 3B, 3C, 4A, 4B, and 4C.

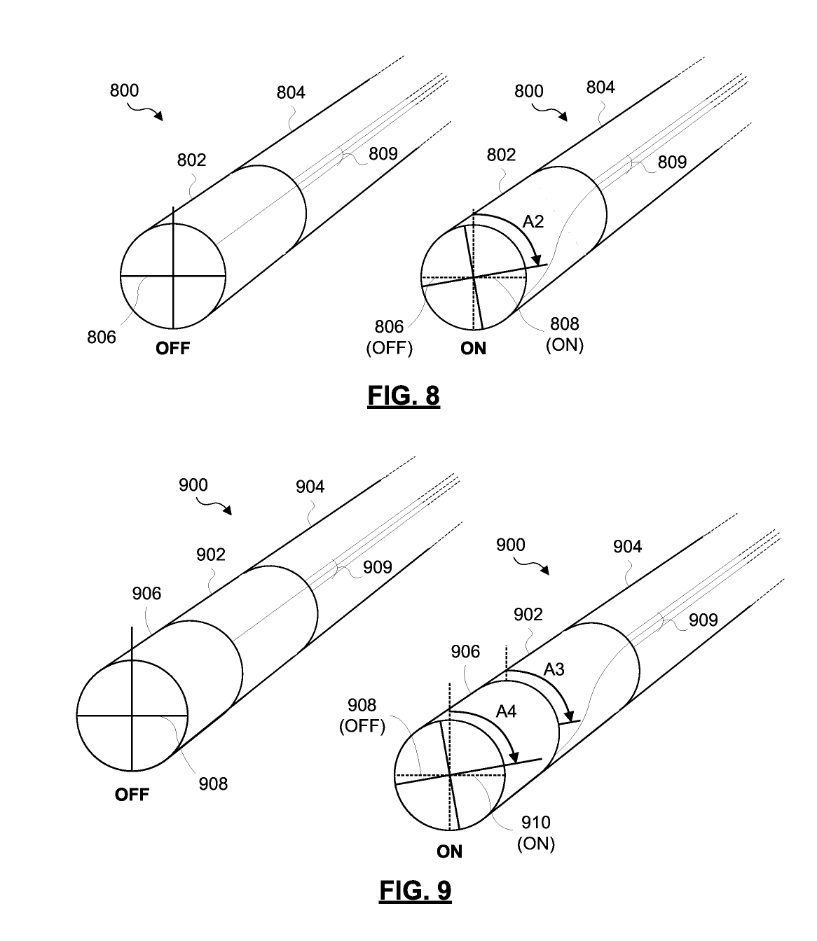

Referring to FIG. 6, an intravascular device 600 is depicted in cross-section as inserted into a vessel 602. Like the intravascular devices depicted in FIGS. 5A, 5B, and 5C, the intravascular device 600 includes a static portion 604 and a dynamic portion 606 that includes one or more artificial muscles. Also depicted in FIG. 6 are two sets of cross-hairs: vessel cross-hairs 608 illustrating the geometric center of the vessel 602, and intravascular device cross-hairs 610 that illustrate the geometric center of the intravascular device 600. In many situations, it is desirable to have an intravascular device as close as possible to the geometric center of the vessel into which it is inserted.

As illustrated in FIG. 6, the dynamic portion 606 is in a dormant state on the left and activated on the right. On the left (i.e., with the dynamic portion 606 in its dormant state), the cross-hairs 608 and 610 illustrate a misalignment of the intravascular device 600 relative to the vessel 602. When activated by an activation energy, the dynamic portion 606 forms a plurality of bumps on its outer surface, such as exemplary bump 612. By configuring the artificial muscles in the dynamic portion 606 to form the plurality of bumps, the activation energy can be used to align the intravascular device 600 closer to the geometric center of vessel 602. Thus, when and while the dynamic portion is activated, plurality of bumps causes the intravascular device 600 to be centered (or at least more centered) within the vessel 602. The size of the bumps may be based on the size of the vessel into which the intravascular device is inserted. Thus, the cross-hairs 608 and the cross-hairs 610 are brought into alignment in the figure on the right. The static portion 604 and the dynamic portion 606 may be incorporated into any of the intravascular devices previously discussed above.

As discussed above in connection with FIGS. 5A, 5B, and 5C, the artificial muscles that form the dynamic portion 606 can be positioned at the distal tip of an intravascular device, but may also be a distance away from the distal tip. For example, an intravascular device incorporating one or more components of an optical coherence tomography (OCT) system on its distal end includes the dynamic portion 606 proximate to the distal end so that, when the dynamic portion 606 is activated, the OCT system is centered within the vessel 602 to provide better data at the region of interest. In some instances an OCT core is received within a lumen of the static portion 604 of the intravascular device 600. This ability to center the intravascular device or a portion thereof is beneficial in many other situations as well.

Referring to FIGS. 7A, 7B, and 7C, an additional application of artificial muscles in an intravascular device context is presented. In FIG. 7A, a intravascular device 700 is depicted that has a flexible elongate member 702 and a dynamic portion 704 at its distal end. The dynamic portion has one or more artificial muscles as described in FIGS. 1A, 1B, 2A, 2B, and 2C, and may share other features with the embodiments depicted in FIGS. 3A, 3B, 3C, 4A, 4B, 4C, 5A, 5B, 5C, and 6. The intravascular device is depicted in a dormant state on the top and in an activated state below. As illustrated, when the dynamic portion 704 is in a dormant state, it has a dormant length 706. When activated the dynamic portion 704 has an activated length 708. The difference between the dormant length 706 and the activated length 708 is a length change 710.

In FIG. 7B, an intravascular device 720 is depicted. The intravascular device 720 is similar in many respects to the intravascular device 700 of FIG. 7A. Thus, the intravascular device 720 includes a flexible elongate member 722 having a proximal portion and a distal portion. Like intravascular device 700, intravascular device 720 has a dynamic portion 724. Unlike in the intravascular device 700, the dynamic portion 724 is located in the distal portion of intravascular device 720, but is not located at the distal tip thereof. As depicted at the top of FIG. 7B, the dynamic portion 724 is in its dormant state and has a dormant length 726. When activated, as seen at the bottom of FIG. 7B, the dynamic portion 726 expands so that it has an activated length 728. The activated length 728 is greater than the dormant length 726 by a length change 730.

In both intravascular device 700 and intravascular device 720, the process of activating the dynamic portion to extend the distal end of those intravascular devices by the respective change lengths. When the activation energy is no longer supplied, the distal end reverts to its dormant position. This process of activating and deactivating the dynamic portions in intravascular devices can be performed rapidly. By activating and deactivating quickly, the distal end of intravascular device 700 and 720 can be used like a microscale jackhammer to strike, pulverize, and otherwise remove an obstruction in a vessel.

FIG. 7C depicts an intravascular device 740 that shares some features with the intravascular devices of FIGS. 7A and 7B. Thus, intravascular device 740 includes a flexible elongate member 742 with a distal portion depicted. In the illustrated embodiment, intravascular device 742 includes a dynamic portion 744 at its distal end. Depicted in the top of FIG. 7C, the dynamic portion is in a dormant state, with a dormant length 746. After an activation energy is applied, the dynamic portion 744 contracts to an activated length 748 that is shorter than the dormant length 746 by a length change 750. When the dynamic portion 744 is deactivated it returns to its previous dimensions and can be used to exert a force at its distal end. This can be done repeated to vibrate or strike an obstruction. In some embodiments, the length change 750 may be multiple times larger than length changes 730 or 710. In such embodiments, the length change 750 is adequate to eliminate the need for a separate pullback device at the proximal end of the intravascular device. In some instances, this is accomplished by having a series of actuators arranged along the length of the intravascular device, with the actuators being activated individually or collectively.

Many modifications can be made to intravascular devices 700, 720, and 740 that are within the scope of this disclosure. For example, in each intravascular device, the artificial muscle materials may be selected so that, rather than axially expanding when activated, the dynamic portions axially contact. Or, rather than axially contracting when activated, the dynamic portions axially expand. Additionally, the dynamic portions in FIGS. 7A, 7B, and 7C may be used an intravascular devices with the morphable tips of FIGS. 4A, 4B, and 4C and/or the dynamic portions depicted in FIGS. 5A, 5B, 5C, and 6. For example, an intravascular device within the scope of this disclosure may have the dynamic portion 606 of FIG. 6 to center the intravascular device within a vessel, the dynamic portion 724 of FIG. 7B to rapidly move the distal end of the intravascular device back and forth, and the morphable tip 414 of FIG. 4 to better shape the distal end for chiseling through an obstruction. Some embodiments may also have an actuator segment 304 as depicted in FIG. 3A, 3B, or 3C. All such combinations are within the scope of this disclosure.

Referring to FIG. 8, a perspective view presents an intravascular device 800 with a torsional actuator segment 802 situated at the distal tip of a flexible elongate member 804. In the depicted embodiment, the torsional actuator 802 is made from carbon nanotube yarn, but in other embodiments torsional actuator 802 may be made from electroactive polymer. On the left of FIG. 8, the intravascular device 800 is depicted with the torsional actuator 802 in a dormant state. A set of axes 806 is depicted at the distal end of the torsional actuator segment 802, and depicts the orientation of the torsional actuator 802 in the dormant state. Additionally, a plurality of wires 809 is included. The plurality of wires 809 extends through the flexible elongate member 804, attaching to a controller on a proximal end and to torsional actuator 802 and/or other devices incorporated in intravascular device 800. The wires 809 may be positioned inside lumens running the length of flexible elongate member 804 and the torsional actuator segment 802.

On the right of FIG. 8, the intravascular device 800 is depicted with the torsional actuator 802 in an activated state. The activation energy applied in the activated state causes rotation in the torsional actuator 802 such that a distal end of the torsional actuator 802 rotates relative to its dormant set of axes 806. As depicted, the total rotation is an angle A2, which as depicted is about 80 degrees in the illustrated embodiment. In other embodiments, the total rotation as described by angle A2 is significantly greater and corresponds to a greater activation energy. Generally speaking, the angle A2 is between about 10 degrees and 1080 degrees. To this end, the characteristics of the artificial muscle or muscles (length, activated or unactived state shapes, applied voltages, materials, etc.) used in the torsional actuator 802 may be selected based on the desired rotation. As depicted, both the torsional actuator 802 and the flexible elongate member 804 are solid. However, in some embodiments the torsional actuator 802 and/or the flexible elongate member 804 have one or more lumens extending part way or all the way therethrough to allow electrical connectors and other components to be positioned therein.

FIG. 9 depicts an intravascular device 900, which is similar to intravascular device 800 of FIG. 8 in several ways. Intravascular device 900 includes a torsional actuator segment 902 coupled to a flexible elongate member 904. But while, the torsional actuator 802 of intravascular device 800 is configured at the distal end of intravascular device 800, intravascular device 900 includes a distal tip portion 906 extending distally beyond the actuator segment 902. The torsional actuator segment couples the tip portion 906 to the flexible elongate member 904. On the left side of FIG. 9, the intravascular device 900 is depicted in a dormant state, with a solid-lined set of axes 908 depicting the orientation of the distal portion 906 while in the dormant state. Additionally, a plurality of wires 909 is included. The plurality of wires 909 extends through the flexible elongate member 904, attaching to a controller on a proximal end and to torsional actuator 902 and/or other devices or functional segments incorporated in intravascular device 900. The wires 909 may be positioned inside lumens running the length of flexible elongate member 804 and the torsional actuator segment 802.

On the right of FIG. 9, the intravascular device 900 is depicted in an activated state. When activated, the artificial muscles of the torsional actuator segment 902 cause the distal end of the segment 902 to rotate relative to the proximal end of the segment by an angle A3. As depicted in the illustrated embodiment, angle A3 is about 80 degrees. In other embodiments, the total rotation described by A3 may be more or less than 80 degrees. Generally speaking, the angle A3 is between about 10 degrees and 1080 degrees. To this end, the characteristics of the artificial muscle or muscles (length, activated or unactived state shapes, applied voltages, materials, etc.) used in the torsional actuator 802 may be selected based on the desired rotation. As discussed in connected with the intravascular device 800 of FIG. 8, intravascular device 900 may include a plurality of lumens extending part way or all the way therethrough to allow electrical connectors and other components to be positioned therein.

As discussed, the distal end of the torsional actuator 904 rotates about 80 degrees. The rotation of the distal end of the torsional actuator 902 causes the distal tip portion 906 to rotate by an angle A4. Angles A4 and A3 are approximately equal in some instances. A dormant set of axes 908 is depicted in dashed lines to indicate the orientation of the distal tip portion 906 in the dormant state, while an activated set of axes 910 depicts the orientation of the distal tip portion 906 in the activated state in solid lines.

In some embodiments, the distal tip portion 906 is also a torsional actuator segment. In such embodiments, the distal tip portion 906 is activated independently of the torsional actuator segment 902. Thus, either distal tip portion 906 or torsional actuator 902 may be actuated in turn, or both may be actuated at the same time. Having the two torsional actuators rotating in the same direction may allow for an increase in the total range of rotational, while having them rotate in opposite direction may allow for increased control or stiffness of the distal portion of the intravascular device 900.

In some additional embodiments, the distal tip portion 906 includes a morphable tip, like morphable tips 404, 414, and 424 of FIG. 4. In one such embodiment, an intravascular device 900 includes a morphable tip 404 as its distal tip portion 906. The morphable tip 404 is activated to provide an abrasive surface, while the torsional actuator 902 is repeatedly activated and deactivated to oscillate the tip.

As described above in connection with intravascular device 800 of FIG. 8, embodiments of intravascular device 900 may include a plurality of lumens. For example flexible elongate member 904 may include a plurality of lumens that hold wires running from a proximal end of the intravascular device to the torsional actuator or actuators to supply them with activation energy. Flexible elongate member 904, torsional actuator 902, and distal tip portion 906 may have lumens partially or fully extending therethrough.

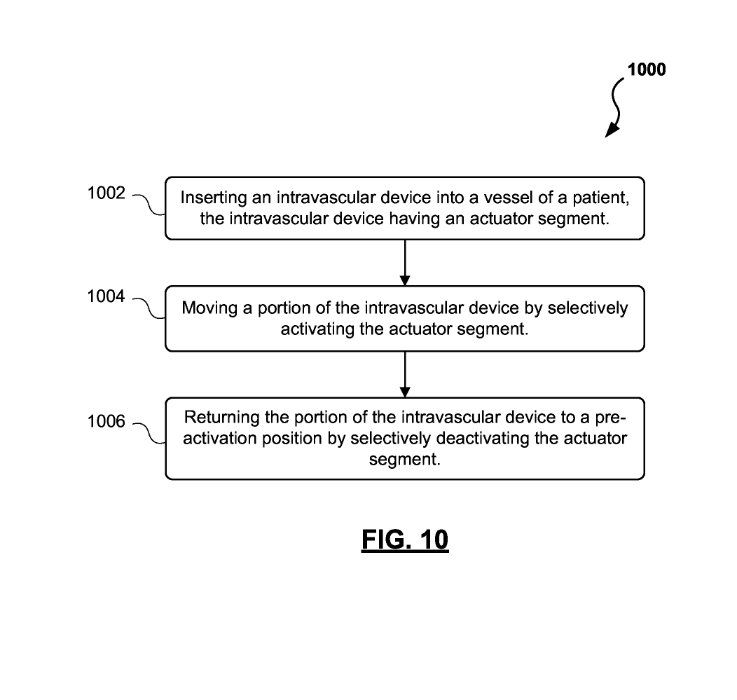

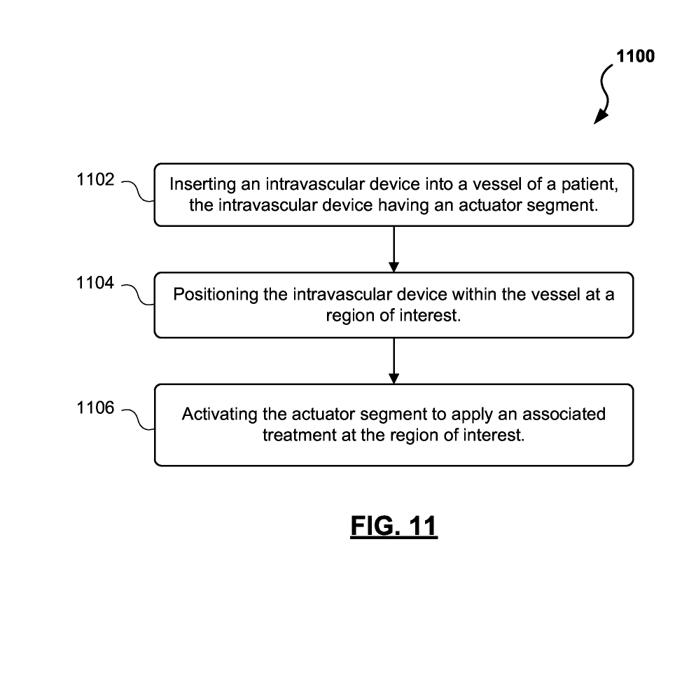

Referring to FIG. 10, a flow chart of a method 1000 for using an intravascular device. Method 1000 begins in step 1002 in which an intravascular device that has an actuator segment is inserted into a vessel of a patient. In step 1004, a portion of the intravascular device is moved by activation of the actuator segment. And in step 1006, the portion of the intravascular device is returned to a pre-activation position by selectively deactivating the actuator segment. In various embodiments of this method, other steps may precede and/or follow some or all of the steps. For example, an additional step may be applying a treatment to the vessel of the patient, prior to returning the portion of the intravascular device to the pre-activation position.

As indicate, in step 1004 a portion of the intravascular device is moved by activation of the actuator segment. Examples of movement include lengthening, contracting, rotating, morphing, expanding, and other such movements as depicted in FIGS. 1A, 1B, 2A, 2B, 2C, 3A, 3B, 3C, 4A, 4B, 4C, 5A, 5B, 5C, 6, 7A, 7B, 7C, 8, and 9. Combinations of such movements can also be performed in step 1004. For example, moving a portion of the intravascular device by selectively activating the actuator segment includes activating a morphable tip, like morphable tip 404 of FIG. 4A, and oscillating the morphable tip using actuator segments like actuator segment 304 of FIG. 3C. In another embodiment, the activated morphable tip is rotated as described in FIGS. 8 and 9. The movements may be part of a positioning process, a data gathering process, a treatment process, and/or other processes performed by an operator of an intravascular device for the benefit of a patient.

To better understand how the method 1000 may be performed, reference is now made to intravascular device 900 of FIG. 9 to provide an example of method 1000. A doctor carefully guides the intravascular device 900 into place within a patient (step 1002). The intravascular device 900 includes an imaging component within the distal tip portion 906. In order to better image the entire region of interest, the imaging component should be rotated about 360 degrees. The doctor uses a controller that provides an activation energy to the torsional actuator segment 902 of the intravascular device. This activation energy is a voltage that is gradually increased so that the torsional actuator 902 rotates through about 360 degrees, while the imaging component captures imaging data (step 1004). The imaging component within the distal portion 906 is rotated gradually to gather the desired data, while the flexible elongate member 904 does not rotate. In some embodiments, the distal portion 906 may include a window, with the window being rotated to image the region of interest. After the distal portion has rotated through the required degrees of rotation, the activation energy is removed and the distal portion 906 of the intravascular device 900 returns to the position it was in prior to activation (step 1006).

FIG. 11 is a flow chart of an additional method for using an intravascular device. As part of method 1100, step 1102 may be performed when a doctor inserts an intravascular device into a vessel of the patient. The intravascular device includes at least one artificial muscle segment configured to provide a treatment to the patient. In step 1104, the doctor positions the intravascular device in or to provide a treatment at a desired region of interest. The treatment may be a material removal treatment that requires the oscillation of a distal tip of the intravascular device provided by activating the artificial muscle segment. In step 1106, the doctor activates the artificial segment to apply an associated treatment at the region of interest. Other steps may occur before, in between, and after steps 1102, 1104, and 1106 without departing the scope of this disclosure.

To better understand method 1100, reference is made to the intravascular device 720 of FIG. 7B that has a dynamic portion 724. As discussed above, the dynamic portion 724 includes at least one artificial muscle, that when activated lengthens a distal portion 722 of the intravascular device 720. The intravascular device also includes the dynamic portion 606 described in FIG. 6. The doctor inserts the intravascular device 720 into a vessel of a patient (step 1102). The doctor guides the end of the intravascular device 720 to a region within the patient that has a certain condition, such as an obstruction and activated the dynamic portion 606 to better center the intravascular device (step 1104). While the device is in this position, the doctor uses a controller to rapidly activate and deactivate the dynamic portion 724 of the intravascular device 720 to drive the distal end of the distal portion 722 through the obstruction. In some embodiments, the intravascular device 720 further includes a morphable tip, such as the morphable tip of intravascular device 410 in FIG. 4.

Persons skilled in the art will also recognize that the apparatus, systems, and methods described above can be modified in various ways. Accordingly, persons of ordinary skill in the art will appreciate that the embodiments encompassed by the present disclosure are not limited to the particular exemplary embodiments described above. In that regard, although illustrative embodiments have been shown and described, a wide range of modification, change, and substitution is contemplated in the foregoing disclosure. It is understood that such variations may be made to the foregoing without departing from the scope of the present disclosure. Accordingly, it is appropriate that the appended claims be construed broadly and in a manner consistent with the present disclosure.

What is claimed is:

1. An intravascular device comprising:
a flexible elongate member having a proximal portion and a distal portion,
at least one torsional actuator segment, the torsional actuator segment being coupled to the flexible elongate member and having a first and a second end, wherein the first end of the torsional actuator segment rotates along an axis of the flexible elongate member relative to a second end of the torsional actuator segment when the torsional actuator segment is supplied with an activation energy.

2. The intravascular device of claim 1, wherein the torsional actuator segment is located in the distal portion of the flexible elongate member.

3. The intravascular device of claim 1, wherein the torsional actuator segment is located in the proximal portion of the flexible elongate member.

4. The intravascular device of claim 1, wherein the torsional actuator segment is made from at least one of carbon nanotubes and an electroactive polymer.

5. The intravascular device of claim 1, further comprising a first conductive line and a second conductive line, the first and second conductive lines running the length of the flexible elongate member and supplying electrical activation energy to activate to torsional actuator segment.

6. The intravascular device of claim 1, further comprising an additional torsional actuator segment.

7. The intravascular device of claim 6, wherein the torsional actuator segment and the additional torsional actuator segment are configured to rotate in the same direction.

8. The intravascular device of claim 6, wherein the torsional actuator segment is configured to rotating in a first direction and the additional torsional actuator segment is configured to rotate in a second direction, opposite the first direction.

9. The intravascular device of claim 1, wherein the intravascular device is a steerable catheter.

10. The intravascular device of claim 9, wherein the steerable catheter includes at least one steering actuator element forming part of the distal portion of the flexible elongate member, the steering actuator element causing a bend in the flexible elongate member in a direction corresponding to a position of the steering actuator element.

11. The intravascular device of claim 1, wherein a distal end of the flexible elongate member includes a morphable tip, the morphable tip having a first shape in a dormant state and a second shape in an activated state.

12. An intravascular device comprising:
a flexible elongate member having a distal end and a proximal end;
a morphable tip at the distal end, the morphable tip having a first shape in a dormant state and a second shape in an activated state.

13. The intravascular device of claim 12, wherein the morphable tip is made from an electroactive polymer.

14. The intravascular device of claim 12, wherein the first shape is a rounded shape and the second shape is a pointed shape.

15. The intravascular device of claim 12, wherein the second shape includes a plurality of bumps.

16. The intravascular device of claim 12, wherein the morphable tip has a center portion and edge portions, wherein in the dormant state the center portion extends distally beyond edge portions and wherein in the activated state the edge portions extend distally beyond the center portion.

17. The intravascular device of claim 12, further comprising a torsional actuator segment forming part of a distal portion of the flexible elongate member such that the morphable tip can be rotated through a range of degrees in order to remove material contacting the morphable tip.

18. The intravascular device of claim 12, further comprising a steering actuator segment that allows the morphable tip to be steered.

19. The intravascular device of claim 12, further comprising a longitudinal actuator segment, the longitudinal actuator segment increasing in length along an axis of the flexible elongate member during a first state and decreasing in length during a second state.

20. A method for using an intravascular device, the method comprising:
inserting the intravascular device into a vessel of a patient, the intravascular device having a torsional actuator segment formed from at least one of carbon nanotubes and an electroactive polymer; and
moving a portion of the intravascular device by activating the torsional actuator segment.

21. The method of claim 20, wherein activating the torsional actuator segment comprises applying a voltage to the torsional actuator segment.

22. The method of claim 20, further comprising returning the portion of the intravascular device to a pre-activation position.

23. The method of claim 22, wherein returning the intravascular device to the pre-rotation position comprises ceasing an application of voltage to the torsional actuator segment.

24. The method of claim 20, wherein moving the portion of the intravascular device comprises rotating the portion of the intravascular device.

25. The method of claim 20, wherein moving the portion of the intravascular device comprises translating the portion of the intravascular device.
